(19) United States
(12) Patent Application Publication
(10) Pub. No.: US 2026/0081784 A1
(43) Pub. Date: Mar. 19, 2026

(54) AUTHENTICATION SYSTEM FOR USE WITH PHARMACEUTICALS

(71) Applicant: I-Property Holding Corp., Sarasota, FL (US)

(72) Inventors: Thomas Gering, Sarasota, FL (US); Alexander Stuck, Wettingen (CH)

(21) Appl. No.: 19/395,686

(22) Filed: Nov. 20, 2025

Related U.S. Application Data
(63) Continuation of application No. 16/665,641, filed on Oct. 28, 2019, now abandoned.
Application No. 16/665,641 is a continuation of application No. PCT/US2018/030383, filed on May 1, 2018.
(60) Provisional application No. 62/492,521, filed on May 1, 2017.

(51) Int. Cl. H04L 9/32; G06K 7/10; G06K 7/14; G06Q 10/0832; G06Q 30/018; G06V 30/10; G16H 20/10; H04L 9/06; H04W 12/069; H04W 12/108; H04W 12/65

(52) U.S. Cl. CPC H04L 9/3239; G06K 7/1094; G06K 7/1417; G06Q 10/0832; G06Q 30/018; G16H 20/10; H04L 9/0637; H04L 9/3236; H04W 12/069; H04W 12/108; G06V 30/10; H04W 12/65

(57) ABSTRACT

An authentication system reads at random or in a predetermined sequence a few, typically 3 to 5, unique product identifiers inside the package to decide whether the content is authentic or not. This cross-referencing check does not require a database connection to a central repository or database. The unique product identifiers are produced when pharmaceutical products are manufactured using embossing, for example, and a product sequence is defined by physical attributes and locations of pharmaceutical products in the packaging, the product sequence then being encrypted and used to mark the packaging of the one or more packaging levels with a coding. The coding is used to authenticate the products in the packaging downstream in the supply chain.

### CROSS REFERENCE TO RELATED APPLICATIONS

This application is a continuing application of, and claims priority to, U.S. patent application Ser. No. 16/665,641, filed Oct. 28, 2019, which claims priority to International Application No. PCT/US2018/030383, filed May 1, 2018, which claims priority to U.S. Provisional Patent Application Number 62/492,521, filed May 1, 2017. The above-mentioned patent applications are incorporated herein by reference in their entireties.

### TECHNICAL FIELD

This disclosure relates to the tracking and authentication of products such as pharmaceutical tablets and other elements stored in blisters or similar packaging, to verify the authenticity thereof.

### BACKGROUND

This application is building on earlier inventions and patent applications by the original named Applicant. These earlier applications and patents include U.S. Patent Publication No. 2015/0183257 to Glendenning et al.; International PCT Patent Publication No. WO 2016/172503 to Stuck; International PCT Patent Publication No. WO 2012/011968 to Stuck et al.; International PCT Patent Publication No. WO 2015/103396 to Glendenning et al.; U.S. Patent Publication No. 2014/0255482 to Klocke et al.; and U.S. Pat. No. 8,715,725 to Stuck et al., the entire disclosures of which are hereby incorporated by reference herein in their entireties.

Pharmaceuticals and cosmetics are high-technology products which require very specialized material systems and production procedures as well as very large investments in development and marketing. Because of public safety concerns, authorities place very stringent requirements on the verification and authenticity of such products. Companies therefore have to make huge investments in the tracking and tracing of these products to ensure authenticity. In addition, as these products usually have large sales margins and are distributed globally, it is not surprising that cosmetics manufacturers and pharmaceutical companies suffer from enormous losses due to counterfeiting. The problem has been aggravated by strongly increased sales over the internet, where everything from counterfeit Viagra to false glucose tests is readily available. Furthermore, local and regional regulations are becoming more stringent regarding the authentication process required for distribution of products including pharmaceuticals.

Various governments around the world have taken the initiative in terms of efforts to increase the authenticity of pharmaceutical products in the supply chain. An overview to these various initiatives is provided at: https://www.infosys.com/SAP/collaterals/Documents/pharmaceutical-serialization-guide-to-country-wise.pdf for example. These initiatives can largely be summarized by the term “serialization” in that most of them thrive to put some sort of barcode on the secondary pharmaceutical package (mostly carton boxes) in the supply chain, and having centralized databases function as code repositories that can be called upon in order to “verify” individual packages. For various reasons these serialization attempts fall short of even achieving a reasonable level of authentication certainty, as is well known in the trade.

Track-and-trace features in the pharmaceuticals market have so far been applied to packaging in various forms. For example, holograms, optically variable inks, fluorescent dyes, and other identification features are attached to the packages, e.g., by adhesive tags. Alternatively, such labels are laminated to the carton or are directly applied to the packages. The main drawback of such labels is that they are not an integral part of the tablet and therefore do not provide 100% security. For example, if the authentic product is separated from the package, the package can be refilled with a false product.

Therefore, a primary concern is still the verification of tablets as such, and ensuring that authentic tablets in fact remain in their correct primary packaging, e.g. in the blister into which these were originally filled on the packaging line.

Once this has been ascertained, such primary packaging (blisters) containing authentic tablets should be verified as resting in the correct secondary packaging (typically carton boxes) into which those blisters have been packed originally on the packaging line.

Most governments today only require that secondary and tertiary packaging contains machine readable codes, such as datamatrix codes, which are unique for each package and serve to identify the package along the supply chain (these methods used are typically being identified by the term “serialization”, as described above).

Usually, all such serialization coding has to be stored in central repositories, where a unique package code and all codes on packages within a certain outer package (sub-packages of a larger shipment) are linked and centrally stored and aggregated. This coding then creates a so-called parent-child relationship between different packaging levels. Every time a package is opened, this event should be communicated to the central repository and the unique package code of the individual package that is being opened is invalidated. This step is usually called de-aggregation.

If the sub-packages are then again put into other packages (which often happens in the case of re-packaging by legitimate distributors along the supply chain), a new package code with a new parent/child relationship is generated and stored in the central repository (aggregation). As pharmaceuticals along the supply chain often change hands from producers to wholesalers, to distributors and finally to pharmacies, aggregation and de-aggregation can happen at many places, and at many different locations in different jurisdictions, requiring each time a package is changed to generate new coding and to invalidate the codes previously printed on the packaging.

In order not to impair the flow of goods by serialization, wholesalers are, in many jurisdictions, only required to check if a given code on the outside of a given package is actually valid. If there is no evidence of tampering with the particular package, it is then assumed that all its contents are acceptable. During de-aggregation however, often all internal packaging codes are checked against the database. No effective and efficient product authentication is required by local governmental authorities in most cases in these current supply chain scenarios. This circumstance is opening the door for counterfeiters, who can copy printed product codes and sell the corresponding packages which they manufacture using fake product, if the fake package is sold and read out against the central database before the one provided by the legitimate manufacturer is sold at a pharmacy. Effectively, this system creates a lottery that sees criminal counterfeiters winning in many cases; and even if the fake product comes up for sale too late, it is often just discarded, often with no direct and detrimental legal consequences to the counterfeiters.

One example of an authentication regime from the Applicant's prior patent documents listed above involves the formation of a hidden security feature on a pharmaceutical package according to U.S. Patent Publication No. 2015/0183257 (“the '257 Publication”). As shown in FIGS. 3 through 6 of that reference, to produce the hidden security feature, a diffractive optical grating pattern is embossed into the surface of the PVC blister pack. This takes place as part of a normal in-line pharmaceutical tablet packaging process on a blister-packaging machine. A standard embossing station on the machine, such as that previously designed for embossing batch codes, can be used to emboss the diffractive grating structure. This station has been used to emboss the diffractive features onto the blister packages. Such an embossing station can include heating units to help control the embossing process.

If desired, an overtly visible hologram can be produced during the same manufacturing process that can be seen brightly in sunlight or white artificial lighting. Another option is to emboss a 2D or 3D barcode into the surface on the material as part of the same process that can be read by a commercial scanner. In both cases the hidden security feature is only revealed when the aluminum foil is selectively removed and a laser pointer is directed in the specific location needed. Thus, a combination of an overt security feature and a hidden security feature are provided in that reference, which will lead most counterfeiters to replicate only the overt information.

Tooling for the embossing process described in the '257 Publication may be manufactured by applying the diffractive structure needed into a hardened tool steel insert or onto other conventional types of embossing tools, such as those made of nickel and similar materials. When used, the hardened steel insert performs the embossing and can hence last for millions of embossing cycles. However, the hardened steel insert operates with functionality identical to that of conventional embossing equipment so that hundreds or thousands of pharmaceutical tablets and packages can be generated per hour of machine operation. The steel insert may also reveal the hidden security feature when a laser pointer is deflected off the relevant embossing surface and when the more complex (multi-level security) hologram includes a hidden security feature. Although the specific holograms produced by the different tools or inserts are different, both are configured to define overt and (optionally) also covert/hidden security features into the structure embossed into the pharmaceutical packaging. It is desired to further improve upon this design that uses overt and hidden security features.

Thus, it would be desirable to improve the authentication system and processes used with packaged goods such as pharmaceuticals.

### SUMMARY

With the present invention, a true authentication system is provided across the entire pharmaceutical supply chain based on physical authentication of the pharmaceutical product by use of truly random coding which serves as a basis for all codes during aggregation and any de-aggregation process along the supply chain. At any given point this inventive scheme allows to authenticate any given package and the sub-packages contained within without the need to access any external database and with a pre-determined level of authentication confidence. In combination with state-of-the-art blockchain theory known to skilled artisans, the authentication system forms random code-based data blocks and uses those blocks as the basis for a blockchain functionality along a manufacturer's supply chain.

Applicant's inventive system is based on the fact that during pharmaceutical production most products, such as tablets and capsules, undergo a true randomization process, as they are processed in industrial tumblers and coated in large quantities in subsequent coating stations. If small variations and subgroups of these products, such as, but not limited to tablets, can be identified and distinguished from each other in production, then the distribution of these subgroups within a given primary package (such as a blister or also a tablet bottle and the like) is a true random number which does not change along the individual package's path through the supply chain. This true random number combined with overt information can then be printed on the primary and secondary package (i.e. the blister or tablet bottle or the paper box which holds blisters for sale). The number can be either directly printed or more likely crypto-graphically secured on the primary and secondary package. Whatever the coding sequence, the result is still a true random number, which is linked to the physical content of the package and can be physically authenticated.

With the true random number being combined with overt information, such an arrangement may help foil counterfeit attempts because the counterfeiter may copy only the overt feature without realizing that a covert feature (the true random number and/or the physical arrangement of tablets/capsules in a primary package) is also present.

In one embodiment of the invention, a blister contains 15 tablets in a given sequence and 3 of these blisters are packed together in a secondary package onto which a barcode is printed on the outer side. Each blister may (but does not have to) contain a printed, laser formed or embossed code that stores the information about its particular tablet sequence. In the case of embossing or laser forming, this would preferably be done at the bottom of the blister in the region where often the manufacturing or batch numbers are embossed. If a security hologram or other optical contrast mechanism is embossed into the same region, the printed, laser formed, or embossed code is visible to conventional camera equipment such as the cameras integrated into smart phones. If the blister is authenticated, using self-authentication (reading the tablet sequence of the blister with a 2D camera and checking it against the embossed, printed or laser formed code on the blister) this inventive solution has the advantage that it only requires taking one single photograph for self-authentication, i.e., the photo will identify the physical tablet sequence and simultaneously the printed/embossed/laser-formed code and a comparison is being made immediately. It is not necessary to turn the blister and read the printed code on the backside in such an authentication according to one embodiment.

In any case, however, the printed code on the outside (secondary) package may contain all information about the individual tablet positions in the blisters contained therein. To authenticate the tablets with a given statistical certainty, in some embodiments it can be sufficient to identify the correct positions of a subset of tablets within a blister and the secondary package.

In one embodiment of this invention, a secondary package contains 3 blisters of the same medication and each of the blisters has 10 tablets packed into it, and there are 2 distinguishable tablet types (for example each tablet has 2 different sides, one containing a logo and one not). An image of a blister is taken with a camera (for example a smartphone), and the sequence of tablets in this particular blister is identified. In order to reach a given level of authentication confidence it is actually not necessary that the entire sequence of the photograph matches the entire sequence of this blister as recorded on the secondary package, a subset of matches is sufficient for reliable authentication. Statistics says that if 6 out of the 10 tablets sit at the correct location with the correct orientation (up versus down) in the blister, the probability that the blister is authentic is higher than 99.9%. The inventive authentication system thus is quite stable in terms of errors or disturbances, as these might occur during transport or because of unstable image recognition by the smartphone camera/software.

If even higher authentication levels or confidence is required in the 3 blister and secondary packaging case described above, one can take a second blister and authenticate it in the same way. The probability, or in authentication, certainty (again with only 6 out of 10 tablets being correctly identified), that the package is not authentic is the square of the first probability, i.e. less than 1 in 1,000,000, with 3 blisters authenticated against the secondary package the certainty is 1 in 1,000,000,000.

The same principle also applies to bottled tablets. The difference here is that there is no order in which the tablets are arranged. By actively embossing measurable geometrical differences in the tablets and manufacturing many different types for the same tablet (for example by embossing a bar code onto the tablet as shown in applicant's U.S. Patent Publication No. 2011/0188051), it is again sufficient to take out and measure 5 tablets and reach confidence levels of 99% or more that the bottle and its content are actually authentic.

In most embodiments according to this invention, the relationships between the printed/laser-formed/embossed tablet sequence and the physical content will not be printed in an overt manner, but crypto-graphically secured, thereby providing an additional protective layer.

The authentication system provides for stable authentication at pre-defined statistical certainty levels for tablets and the associated primary and secondary packaging. The authentication is based on truly random coding and allows authentication of a product with a predetermined and high accuracy. With the system and process relying on truly random input information and the information being crypto-graphically secured, it is in fact extremely difficult to successfully counterfeit.

To realize the inventive authentication system in a manufacturer's supply chain, the following inventive products need to be used and integrated into the system. First, certain marking of products such as pharmaceutical dosage forms needs to occur by embossing or the like, as set forth in the prior applications referenced above. Second, certain digital camera technology that can read random coding must be provided on the packaging line, as and when tablets are being packaged. For example, readers such as pOCT or time-of-flight camera technology can be used to help achieve the authentication system of this invention. Third, software applications and products that function as shown in flowcharts described below with reference to FIGS. 1 through 3 must be provided, which are implemented in customer reader or smart phone applications and which can be used for cross-referencing along the supply chain.

In all embodiments, the secondary package (i.e. smallest saleable unit in many markets around the world) is aggregated into a larger box and this box is also provided with a unique identifier, which may be used in yet further embodiments of the authentication system of this invention.

In one specific embodiment of the invention, an authentication system for pharmaceutical products includes product manufacturing equipment that uses embossing technology to form and mark the pharmaceutical products with a coding. The system also includes a packaging line that places the pharmaceutical products in one or more packaging defining one or more packaging levels. In-line reading equipment determines a product sequence defined by physical attributes and locations of pharmaceutical products in the packaging, the product sequence then being encrypted and used to mark the packaging of the one or more packaging levels with a coding. The system further includes scanning equipment at a point in a supply chain downstream from the packaging line, the scanning equipment including a camera that images the codings of the pharmaceutical products and of the one or more packaging levels and a processor that compares the codings to one another to authenticate whether the pharmaceutical products are genuine. The product manufacturing equipment marks the pharmaceutical products without adding marking products such as inks to the pharmaceutical products. The encryption of the product sequence is achieved using public-private key. The authentication of whether the pharmaceutical products are genuine is achieved without causing the scanning equipment to communicate with a central data repository away from the supply chain.

### BRIEF DESCRIPTION OF THE DRAWINGS

Various additional features and advantages of the invention will become more apparent to those of ordinary skill in the art upon review of the following detailed description of one or more illustrative embodiments taken in conjunction with the accompanying drawings. The accompanying drawings, which are incorporated in and constitute a part of this specification, illustrate one or more embodiments of the invention and, together with the general description given above and the detailed description given below, serve to explain the one or more embodiments of the invention.

FIG. 1 is a schematic flow chart showing a series of steps for production and aggregation of an encrypted hash, which may be implemented by a computer having a processor and similar equipment loaded with software, in accordance with one embodiment of the present invention.

FIG. 2 is a schematic flow chart showing a series of steps for authentication and verification of a sequence using an encrypted hash, which may be implemented by a computer having a processor and similar equipment loaded with software, in accordance with one embodiment of the present invention.

FIG. 3 is a schematic flow chart showing a series of steps for authentication using symmetrical encryption, which may be implemented by a computer having a processor and similar equipment loaded with software, in accordance with one embodiment of the present invention.

### DETAILED DESCRIPTION

In accordance with one embodiment, the authentication system operates along the supply chain in the following manner.

Instead of only checking if the latest outside code is valid and included in a central database repository, as is typical in conventional systems, the authentication system reads at random or in a predetermined sequence a few, typically 3 to 5, unique product identifiers inside the package to decide whether the content is authentic or not. Again, this cross-referencing check does not require a database connection to the central repository and it can therefore be performed with relatively simple readers or smart phone software applications anywhere in the world while achieving security levels that are actually higher than the ones achievable by use of central repositories—as their sub-systems are having to deal with their own error events (readers not working reliably, database errors, aggregation errors, internet connection problems, etc.). In many of those events the central repository systems have no other choice but to send product back to the manufacturer as it cannot be reliably authenticated when in the supply chain. The authentication system of this invention eliminates such issues, as any package in the supply chain is inherently secure and can be reliably authenticated when it is already in the supply chain without the need of going back to the central repository for any information.

In this regard, because the verification is within the blister pack itself in an example where the package is a blister pack, no database access or database management is needed for performing the authentication or verification of the blister pack. More specifically, verification codes and data do not need to be sent to external databases for verifying the authenticity of a blister pack and its contents. As a result, verification of the blister pack occurs immediately upon imaging and detection of the unique product identifiers, which means that packages such as blister packs can be verified as authentic within one second. Conventional systems reliant on communication with external servers do not compare favorably with the time efficiency offered by this anti-counterfeiting and authentication process.

The authentication system is hierarchic and can be extended to any level of packaging. A user always checks the code on the outer package and compares it with a small statistically-significant sample of the codes within to reach a desired level of authentication. In terms of information every package forms a block, which is cryptographically secured and some or all packages on the same level together with their printed (or naturally measurable) codes form a blockchain, which if packaged together in a larger box itself then forms a secured block on the next higher packaging level. This inventive system fully circumvents the problem of aggregation/de-aggregation, as for de-aggregation now only the codes of the outer packages must be destroyed, while aggregation now always happens the same way by creating a secured block. The specifics of how each block is cryptographically secured can vary widely based on the embodiment, as will be readily understood by those skilled in the art. As at the lowest level (the product or blister) the product code is random and linked to the unique physical properties of an individual product, the product itself can not only be tracked and traced by the system through the supply chain from manufacture to point of distribution, but also effectively authenticated with a high degree of confidence.

In one embodiment of the invention, coding created by the authentication system is cryptographically linked to the central repository coding, which will nevertheless be necessary because of governmental requirements in target markets. However, the benefits of reliable authentication at any point in the supply chain are still realized in such circumstances.

One example of such a set of governmental regulations that the embodiments of this invention help improve compliance with is described in the '257 Publication. In this regard, the embodiments described herein are potentially useful also in markets where—to date—the majority of dosage forms are shipped in bulk and re-packaged along the supply chain. Although there is a certain trend at least in the United States to introduce blister packs, bulk-deliveries and re-packaging will still be possible after introduction of new serialization policies, which in the U.S. was set to begin with a California state law that was to originally become effective in 2015. Under this law, the so-called electronic pedigree requires a “record, in electronic form containing information regarding each transaction resulting in a change of ownership of a given dangerous drug, from sale by a manufacturer 14, through acquisition and sale by one or more wholesalers, manufacturers, or pharmacies, until final sale to a pharmacy or other person furnishing, administering or dispensing the dangerous drug. The pedigree shall be created and maintained in an interoperable electronic system, ensuring compatibility throughout all stages of distribution.” See California Business and Professions Code Section 4034.1.

The rules regarding the pedigree do not, however, dictate the technological implementation of identifiers put onto pharmaceutical packaging, such as blister packs or bulk packaging. In fact, the California government's written summary on e-pedigree laws says specifically: “The unique identifier . . . on each saleable container of prescription drugs will most likely be carried on either a 2D bar code or an RFID chip placed on the saleable unit by the manufacturer 14. The California Legislature has not mandated these specific technologies, but they are the two methods that have been identified that could meet the requirements of the legislation.” It is noted that this California law (and many other proposed U.S. state laws that were set to go into effect after 2015) was superseded before enactment by a corresponding U.S. federal law entitled the Federal Drug Supply Chain Security Act (DSCSA). Under the DSCSA, any packaging or repackaging of pharmaceuticals must include affixed or imprinted product identifiers on each package so that genuine packages can be tracked through commerce using electronic monitoring and records gathering systems for a number of years. The authentication system proposed in this application will help manufacturers and suppliers in achieving compliance with the DSCSA and other local laws and regulations that may come into effect regarding this field.

In the field, if there is any suspicion that a package or some barcodes have been tampered with, when using the authentication system, it is possible to go down to any packaging level deemed necessary reading aggregated coding to evaluate authenticity of the product. In the authentication system such reading of aggregated coding on various packaging levels can even go down to the product level and thereby authenticate the contents of any package. Therefore, the hierarchical authentication system of this invention is almost entirely immune against simple copying of some or all barcodes on the packages by counterfeiters, which is deemed the most probable attack scenario based on currently-known counterfeit activity.

This approach of scanning the contents of a package also implicitly provides tamper evidence at the same time because it authenticates the content of the packages. The authentication system therefore allows manufacturers to avoid use of any expensive security tamper evidencing labeling of packages, additional tamper evidencing structures, etc., while at the same time gaining considerable additional authentication capabilities.

One implementation embodiment of the authentication system of this invention is described as follows.

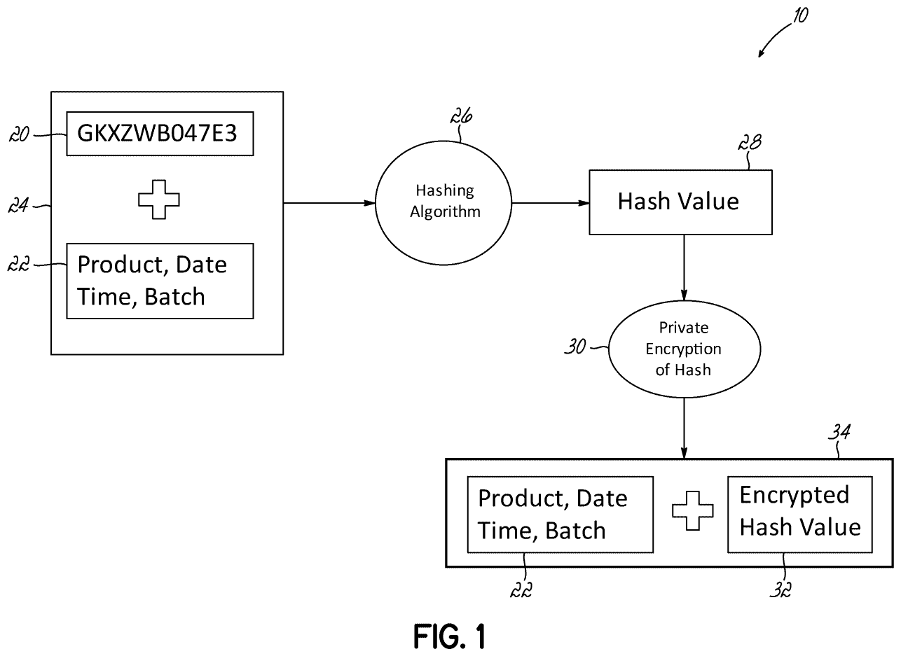

A blister containing 10 tablets has a simple random tablet code sequence of up and down sides, converting to 0's and 1's in a data collection matrix. With 10 tablets, 1024 code sequences are possible. The printed code contains open information, (tablet type, lot number, date and time of manufacturing, manufacturing line, etc.) plus a cryptographic 7-digit hash code, which is generated by cryptographically combining the actual binary tablet code sequence with the open information. As the hash has 7 digits, 10 million hashes are possible, however, out of these only 1024 are possible correct hashes. A reader who knows the cryptographic scheme used (meaning it has the correct keys stored in it) can then immediately decide by simply reading the open information and the hash, if the hash can actually be a true hash or not. In a further step, for true authentication the actual physical binary sequence can be checked. One version or embodiment of these functions is described below with respect to FIGS. 1 through 3.

An embodiment creating higher level statistical testing is the following. There are n secondary packages (blisters packed in a carton, each carton having a unique product identifier printed on the outside) packed in one larger box, which has also a printed unique product identifier on its outside. A simple algorithm is assigning a binary number (or any other number for that matter) to each unique product identifier of each carton (for example, if the checksum of the unique product identifier is between 0-4 or between 5-9 gives a 0, resp. 1). Then the packages in the box form a binary (or any other) sequence depending on their position in the box. This sequence can again be encrypted and hashes or similar checks printed on the outside of the box. It is possible to predetermine a given (if possible secret) subsequence and have a shorter code for fast crosschecks. For example, if n=125 in a box, with 5×5×5 cartons a natural subset might be to simply read the topmost 5 codes on the cartons and their corresponding sequence to determine if the content inside the box is likely to be genuine and has not been tampered with.

Now turning to the flowcharts of authentication operations possible with the authentication system of the present invention, in FIG. 1, the production of an encrypted hash is shown as process 10. The encrypted hash is produced during initial manufacturing/packaging at the beginning of the supply chain (with product manufacturing equipment that uses embossing technology), or during aggregation in the supply chain. The actual product sequence or subsequence 20 (defined by physical attributes and locations of product in a blister pack 24, for example) and open information 22 such as the product, date time, and batch of the blister pack 24 are combined using a hashing algorithm 26 to form a hash value 28. This hash value 28 then goes through step of a private encryption of the hash 30 to form what is printed on the outside of the box 34, including the open information 22 as well as the encrypted hash value 32. From this encrypted hash value 32, the authentication of the package 24 and its contents can be performed in accordance with the invention.

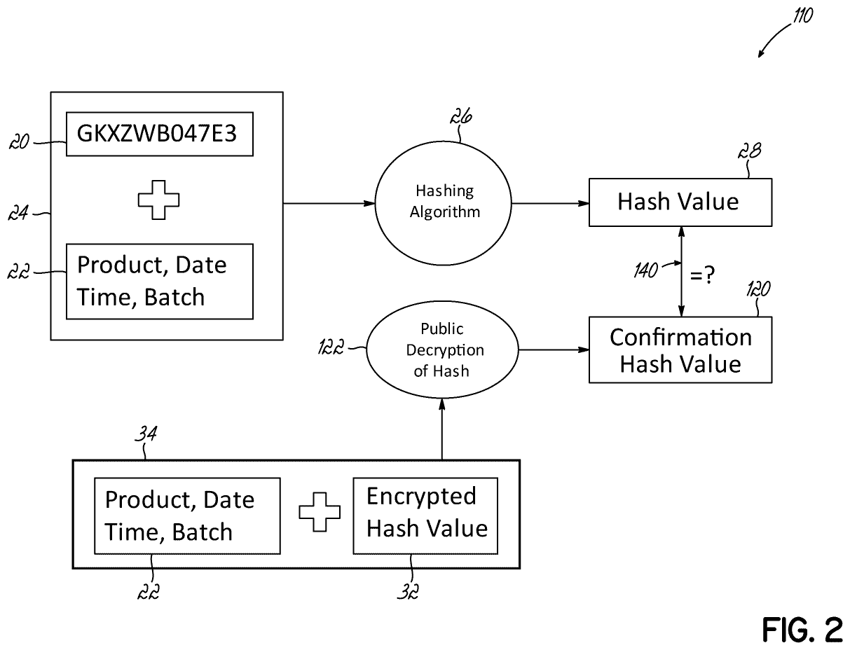

In FIG. 2, the authentication and verification of the sequence using an encrypted hash is shown in flow chart form as process 110. The top portion of this Figure is the same as FIG. 1, e.g., the actual product sequence or subsequence 20 in the package 24 and open information 22 such as the product, date time, and batch are combined using a hashing algorithm 26 to form a hash value 28. This hash value 28 is compared to a confirmation hash value 120 determined using a public description of the hash 122 applied to the information printed on the outside of the box 34. If the hash values 28, 120 agree at comparison step 140, then the package 24 and its contents are authenticated. The authentication is performed using scanning equipment that only requires a camera and a processor, which may be found in modern smart phones for example.

In this embodiment, the hash is actively encrypted at hashing algorithm 26 with a private key and decrypted at the public decryption of hash 122 with a public key. This step can be omitted if a secret hashing algorithm is used or a known hashing algorithm is altered in a secret way, for example by using so-called salts and peppers or similar security schemes. Authentication of the product sequence in this particular example then happens by repeating the same hashing procedure and decrypting the printed hash with a public key. If both hashes are identical, the product sequence has not been tampered with, as set forth above. This method does not rely on remote database communication to conduct the necessary authentication.

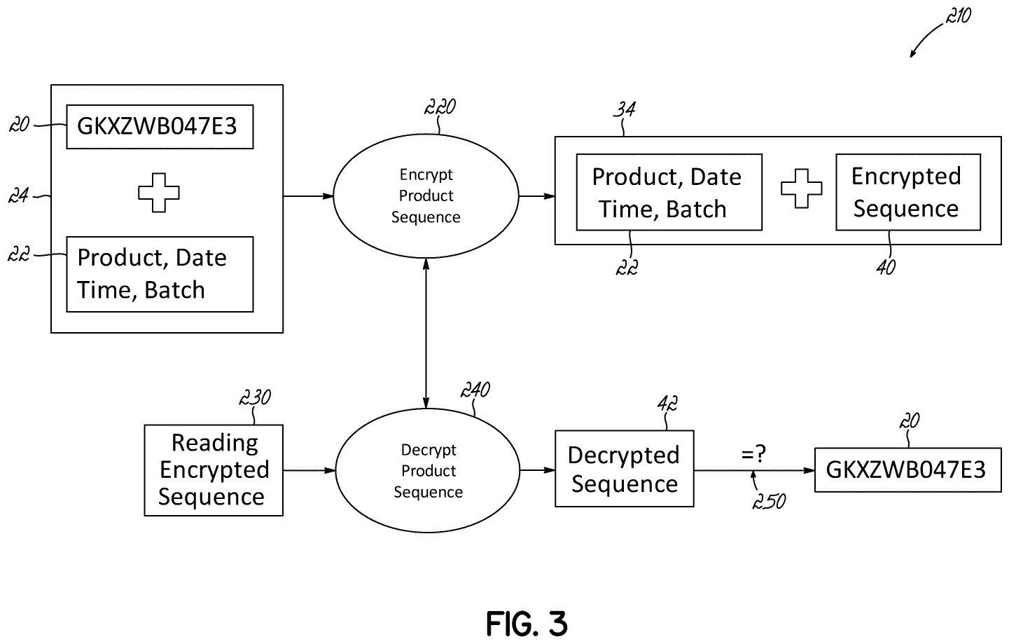

In a further embodiment of the invention, the product sequence is encrypted using a symmetric key, the full encrypted sequence or a predetermined part of it is then printed on the outside box, or the back of the blister. For example, FIG. 3 shows a flow chart summarizing this embodiment of authentication using symmetrical encryption, as process 210. The top portion of the diagram shows the production of the package 24, in which the actual product sequence or subsequence 20 is combined with open information 22 such as the product, time, date, and batch, and then the product sequence is encrypted (at step 220) to form what is printed on the outside of the box 34, e.g., the open information 22 and the encrypted sequence 40. The bottom portion of the diagram shows what a user does to authenticate, including reading the encrypted sequence at step 230, using a public/private key scheme to decrypt the product sequence at step 240, resulting in a decrypted sequence 42, and then comparing the decrypted sequence 42 to the actual product sequence 20 at comparison step 250 to determine if the product and package are authentic. When these elements match, the product and package are authentic.

For authentication, the symmetric key to decrypt the sequences is sent to authorized receivers in encrypted form, using private/public keys to protect the symmetrical key from attacks. This exchange of information is indicated by the double-headed arrow shown extending between steps 220 and 240 in the flow chart of FIG. 3.

Moreover, multiple structures may be used to provide additional optical contrast or authentication/verification of the pharmaceutical and/or packing, as described in U.S. Pat. No. 8,715,725 to Stuck et al. In this regard, additional security features may also be added in combination with those identified in the embodiment above.

As briefly described above, a generalized example of one or more additional security features on the same blister pack as the security/authentication features described above is outlined as follows. To this end, the blister pack may also include one or more of: a 2D barcode, a hologram (which may include detectable defects and/or irregularities unique to the embossing tooling used to form the hologram), and lettering in the form of microtext. The microtext defines depth and/or lateral dimensions between 2 microns to 100 microns. To this end, a microtext may cover a larger area on the blister pack of several millimeters across (e.g., such as a 10 millimeter by 10 millimeter square of microtext). In practice, the micron sized features (microtext) are distributed over this area, but the features need not be connected.

In the virtual world, blockchain-related technologies create blockchains and then distribute identical copies of same on many computers, with the copies being continuously updated and authenticated using various distributed ledger systems depending on the specific blockchain technology being used. In the present invention, the blockchain is physically implemented as the box plus the code on the box, e.g., by using random codes extracted from individual physical product properties.

While the present invention has been illustrated by the description of various embodiments thereof, and while the embodiments have been described in considerable detail, it is not intended to restrict or in any way limit the scope of the appended claims to such detail. Thus, the various features discussed herein may be used alone or in any combination. Additional advantages and modifications will readily appear to those skilled in the art. The invention in its broader aspects is therefore not limited to the specific details and illustrative examples shown and described. For example, the invention described herein is not limited to application on pharmaceutical blisters, but instead can be used for any product and package which allows to generate a reproducible unique random code for each product for example from a picture taken with a camera. The present invention then takes these individual random product codes and forms a block with them. At each level of packaging these blocks then are formed into a physical blockchain which is cryptographically secured at the next level and so on. Accordingly, departures may be made from such details without departing from the scope of the general inventive concept.

What is claimed is:

1. An authentication system for pharmaceutical products, comprising:
product manufacturing equipment that uses embossing technology to form and mark the pharmaceutical products with a coding;
a packaging line that places the pharmaceutical products in one or more packaging defining one or more packaging levels;
in-line reading equipment to determine a product sequence defined by physical attributes and locations of pharmaceutical products in the packaging, wherein the packaging line uses a hashing algorithm to encrypt the product sequence, which is then used by the packaging line to mark the packaging of the one or more packaging levels with a coding, with this coding marked on the packaging including the encrypted product sequence;
scanning equipment at a point in a supply chain downstream from the packaging line, the scanning equipment including a camera that images the codings of the pharmaceutical products and of the one or more packaging levels and a processor that compares the codings to one another to authenticate whether the pharmaceutical products are genuine, wherein the scanning equipment is configured to authenticate the pharmaceutical products by reading only a subset of the codings of the pharmaceutical products, the subset comprising between three to five of the pharmaceutical products, and wherein authentication is achieved by comparing the codings of the subset of pharmaceutical products to the coding marked on the packaging to determine whether the pharmaceutical products are genuine,
wherein the product manufacturing equipment marks the pharmaceutical products by the embossing technology, the encryption of the product sequence is achieved using public-private key, and the authentication of whether the pharmaceutical products are genuine is achieved without causing the scanning equipment to communicate with a central data repository away from the supply chain, and
wherein the system is configured to produce a series of packagings each having a coding, the series of packagings and the codings collectively defining a blockchain that can be stored and used for authentication at any point in the supply chain,
wherein aggregation of a group of packagings to be stored in an outer container occurs by forming the blockchain for the group of packagings and cryptographically securing the blockchain for marking on the outer container, and
wherein de-aggregation of a group of packagings stored in the outer container includes only destruction of markings or codings of the outer container but not of the group of packagings.

2. The authentication system of claim 1, wherein the product sequence is encrypted into the coding for the one or more packaging levels using a hashing algorithm.

3. The authentication system of claim 1, wherein the product sequence is encrypted into the coding for the one or more packaging levels using a symmetrical encryption.

4. The authentication system of claim 1, wherein the scanning equipment further includes at least one of:
a smart phone including the camera and the processor;
a parallel Optical Coherence Technology (pOCT) scanner; and
a time-of-flight camera.

5. The authentication system of claim 1, wherein the physical attributes defining the product sequence are generated by the product manufacturing equipment, the product sequence being randomized by placement of the pharmaceutical products containing the physical attributes in the locations within the packaging.

6. The authentication system of claim 1, wherein the packaging levels include blister packs into which the pharmaceutical products are loaded, and one or more outer boxes in which the blister packs are loaded.

7. The authentication system of claim 1, wherein the packaging levels include a pill bottle into which the pharmaceutical products are loaded.

8. The authentication system of claim 1, wherein the step of authenticating whether the pharmaceutical products are genuine with the processor automatically provides tamper evidence, thereby avoiding a need for tamper-evidencing structures to be included with the packaging.

9. The authentication system of claim 1, wherein the coding on the packaging includes overt information in combination with the encrypted product sequence to help foil counterfeit attempts of the packaging.

10. The authentication system of claim 1, wherein the coding on packaging may be reapplied in the supply chain when the pharmaceutical products are repackaged down the supply chain, while maintaining traceable records that can be authenticated for each of the pharmaceutical products that has been repackaged.

11. The authentication system of claim 1, wherein additional security features separate from the coding are applied to the packaging.

12. The authentication system of claim 1, wherein the blockchain is stored in a distributed ledger system and the authentication system uses cryptographic encryption of a hash formed by the product sequence and encrypted and decrypted using public-private key.

13. The authentication system of claim 12, wherein the authentication system combines the product sequence and further open information regarding the pharmaceutical products or packaging into a hash value by a hashing algorithm, the hash value is encrypted using a private key and decrypted with a public key, and the hash value is a 7-digit hash code such that only a selected subset of over 10 million hashes are saved in the authentication system as possible correct hashes, thereby allowing for a further check of authenticity.

14. The authentication system of claim 13, wherein aggregation of a group of packagings to be stored in an outer container occurs by forming the blockchain for the group of packagings and cryptographically securing the blockchain for marking on the outer container.
